(19) United States
(12) Patent Application Publication
(10) Pub. No.: US 2008/0237365 A1
(43) Pub. Date: Oct. 2, 2008

(54) LIQUID DISPENSER

(76) Inventor: Abdul Ebrahim Patel, Essex (GB)

Correspondence Address: BARLOW, JOSEPHS & HOLMES, LTD., 101 DYER STREET, 5TH FLOOR, PROVIDENCE, RI, 02903, US

(21) Appl. No.: 12/060,901

(22) Filed: Apr. 2, 2008

(30) Foreign Application Priority Data
Apr. 2, 2007 (GB) 0706425.6

(51) Int. Cl. A61L 9/00; B65D 8/04

(52) U.S. Cl. 239/34; 220/669

(57) ABSTRACT

A liquid dispenser includes a container 1 for liquid to be dispensed. A strip of sheet material 6 is secured to the container 1. The container 1 is shaped so that the strip of material 6 and container 1 together define an enclosed region in which an air freshening product 5 may be disposed. The dispenser provides for a combined liquid soap dispenser and air freshener which is less costly to manufacture than known arrangements.

### BACKGROUND OF THE INVENTION

1. Field of the Invention

The present invention relates to a liquid dispenser capable of dispensing a liquid soap, hand cream or the like and an air freshener product.

2. Background of the Related Art

Dispensers for dispensing liquid soap and an air freshener product are known. One such arrangement, intended in particular for use in aircraft washrooms, is disclosed in GB 2414466. The dispenser comprises a plastic bottle for liquid soap fitted with a pump action dispenser and a separately moulded housing accommodating an air freshener dispensing arrangement.

The separately formed moulded housing for the air freshener is costly to produce and adds significantly to the overall cost of the end product. The housing is injection moulded, involving the manufacture of an injection moulding tool. Changes to the design of the moulded housing, to suit customer preference, will therefore involve the manufacture of a new, different, injection moulding tool. Further, the injection moulding process will, in practice, often take place at a different geographical location to that where the bottle is produced and it is thus necessary to transport the two components in order to bring them together for assembly and subsequent distribution. Both the bottle and moulded housing are bulky items which cannot be packed efficiently for transportation.

It is an object of embodiments of the present invention to provide a liquid dispenser capable of dispensing liquid soap and an air freshener product which provides the same or similar functionality to that disclosed in GB 2414466, but which can be manufactured at a lower cost.

### SUMMARY OF THE INVENTION

According to the present invention there is provided a liquid dispenser comprising a container for receiving a liquid and a strip of sheet material secured to the container, the container being shaped such that the strip of material and container together define an at least partially enclosed space in which an air freshening product may be disposed.

With this arrangement it is only necessary to provide one moulded part, that is the container. The dispenser is completed by a strip of sheet material. Strips of sheet material, for example a plastics sheet material, can be provided at significantly lower cost than a moulded housing. Strips of material can also be packed extremely efficiently for transportation.

The container, which may be a bottle, may be shaped to define a recess and the strip may be arranged to overlie the recess.

The strip may be perforated and/or may be breathable. Where the strip is perforated the perforations may be arranged to present a decorative design, a word, trade mark or other indicia. The design presented by the perforations may be changed at relatively low cost in order to suit customer preference. To do so it is only necessary to provide a new cutting tool to form the perforations, as compared to providing a new injection moulding tool as for the arrangement of GB 2414466.

An air freshening product may be disposed in the at least partially enclosed space such that air freshener may diffuse from the enclosed space into the surrounding environment.

The air freshening product may be formed from any suitable material. In some embodiments the air freshening product is a material which absorbs or supports an air freshener for subsequent release. It may be a strip of material, such as a plastics, paper, card or other absorbent material. The container may have a recess formed around its periphery and the strip of sheet material may extend around the periphery in the region of the recess. The strip of material may comprise a fastening means to enable it to be secured to the container and/or to enable it to be secured to itself so that it may be fastened around the container. With the latter arrangement, the fastening means may comprise cooperating engageable formations, such as a tab and slit, in the strip.

In one embodiment the container comprises a generally cylindrical bottle. A recessed region, of reduced diameter, extends around a part of the bottle. An air freshening strip is wrapped around the recessed part of the bottle and the strip of material extends around the bottle over the air freshening strip thereby capturing the air freshening strip in a cavity. The strip of material is perforated and/or otherwise breathable enabling air freshener to diffuse from the air freshening strip via the strip of material into the surrounding environment.

The bottle may contain a liquid soap, hand cream or a similar cleansing or cosmetic product. For use in aircraft washrooms and other public washrooms the container may include a formation enabling it to be mounted on a mounting base. The formation may be arranged so that the container may otherwise not stand upright for use, thereby reducing the risk of theft of the container.

### BRIEF DESCRIPTION OF THE DRAWINGS

In order that the invention may be more clearly understood, an embodiment thereof will now be described, by way of example, with reference to the accompanying drawings, of which:

FIG. 1 shows a side view of a liquid dispenser including an air freshener;

FIG. 2 shows a vertical cross-section through the dispenser of FIG. 1;

FIG. 3 shows the same view as FIG. 2, but with the air freshening strip and cover strip removed;

FIG. 4 shows a side view of the air freshening strip of the dispenser of FIG. 1;

FIG. 5 shows a side view of the cover strip of the dispenser of FIG. 1;

FIG. 6 shows a side view of an alternative cover strip;

FIG. 7 shows a side view of a mounting base for the dispenser of FIG. 1; and

FIG. 8 shows a perspective view from above of the mounting base of FIG. 7.

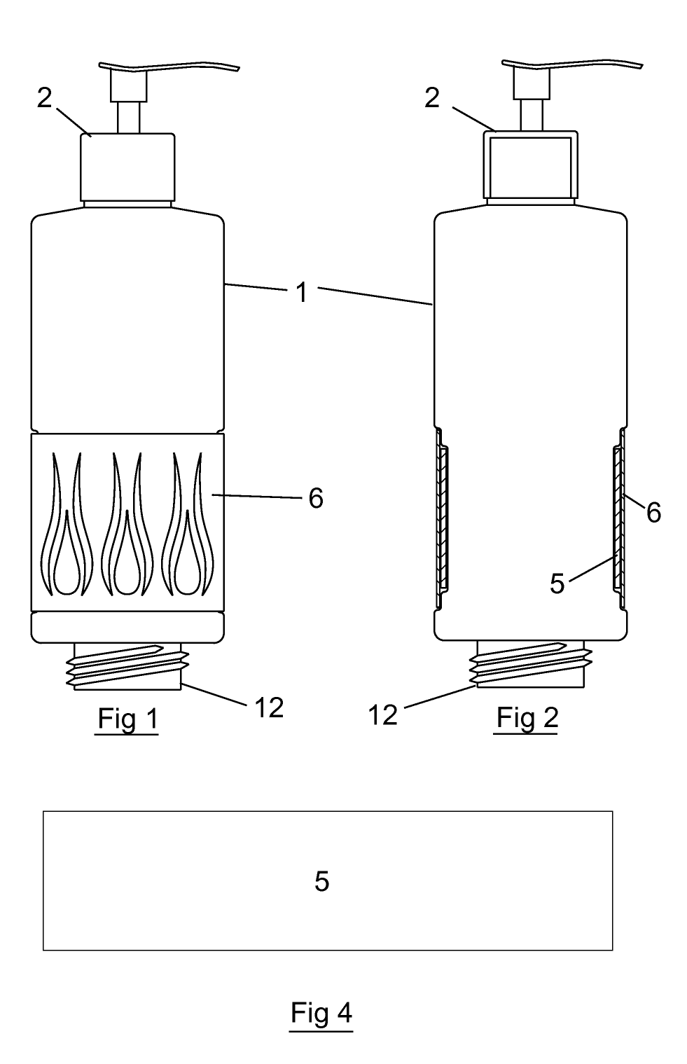

### DETAILED DESCRIPTION OF THE PREFERRED EMBODIMENT

Referring to the drawings there is shown a liquid dispenser, primarily intended for dispensing a liquid hand soap or cream, including an air freshener. The dispenser is particularly intended for use in bathrooms and washrooms and is particularly useful in aircraft washrooms.

The dispenser comprises a moulded plastics bottle 1. The bottle is substantially circular in horizontal (as illustrated) cross-section. At the top of the bottle (again as illustrated) there is a threaded neck to which is fitted a pump dispenser 2. The pump dispenser is of conventional type and therefore need not be discussed further. Other suitable means for dispensing liquid from the bottle could be employed, as appropriate.

At the upper half of the main body of the bottle the diameter of the bottle is substantially constant. Approximately half way down the main body of the bottle its diameter reduces in two steps 3. The diameter increases in two corresponding steps 3 towards the bottom of the bottle. Thus, a recessed region 4 is formed in the outside of the bottle.

The recessed region accommodates an air freshening strip 5 located between the two second steps forming the recess. The air freshening strip 5 is formed by a generally rectangular strip of a flexible sheet material capable of absorbing or otherwise holding and releasing a fragrance or other air freshening substance. Suitable materials include paper, card and plastics materials. The air freshening strip is wrapped around the bottle 1.

The air freshening strip is held in place by a cover strip 6. The cover strip is formed from a generally rectangular strip of flexible plastics sheet material although other suitable sheet materials could be used. The strip is perforated to allow the air freshening substance of the air freshening strip to diffuse into air surrounding the dispenser. Perforations in the strip may form a decorative design or could form a word, trade mark or other indicia. At one end of the strip there is formed a tab 7 and towards the opposite end is a corresponding slit 8 arranged to receive the tab 7. The tab 7 and slit 8 enable the strip to be fastened around the bottle 1. The strip 6 is sized and the tab 7 and slit 8 positioned such that the strip fits snugly around the bottle 1, supported on the first step 3 of the recessed region with the outside of the strip 6 lying substantially flush with the remainder of the bottle 1.

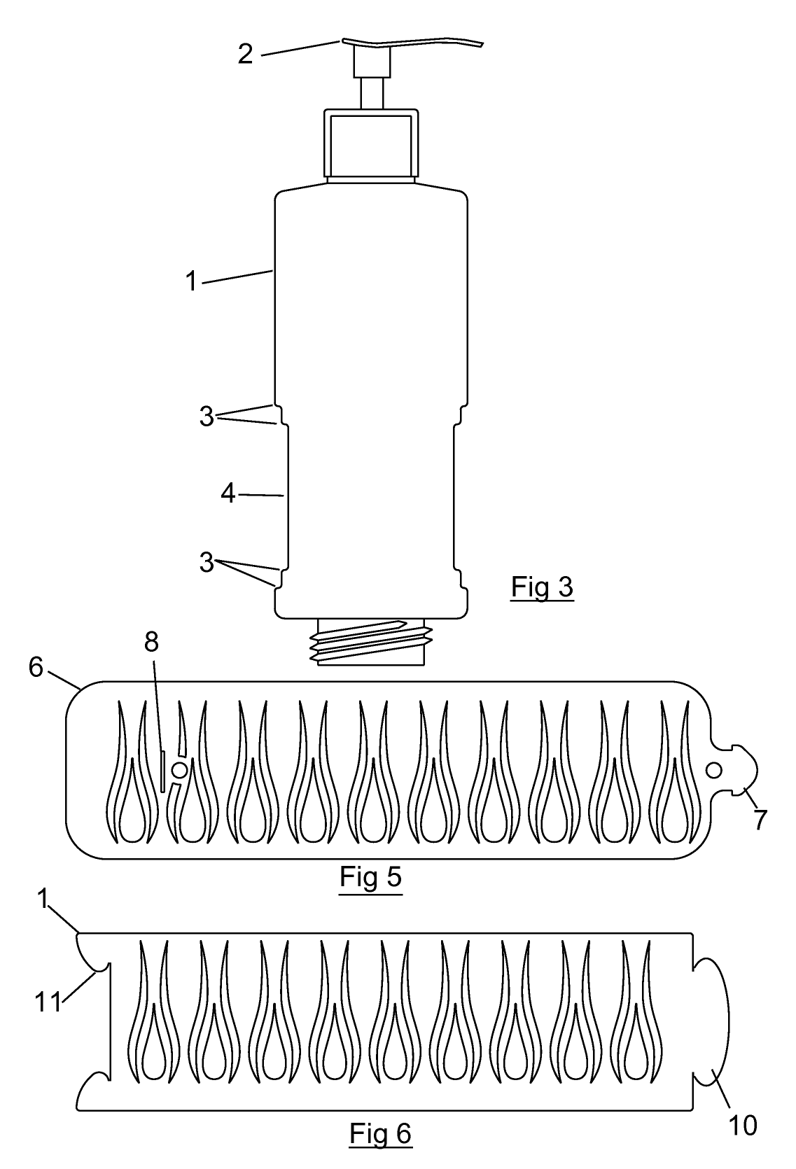

FIG. 6 shows an alternative design of strip of having a tab 10 at one end and a cut-out formation 11 at the opposite end enabling the strip to be fastened end to end around the bottle. Other forms of strip with different forms of fastening are, of course, possible.

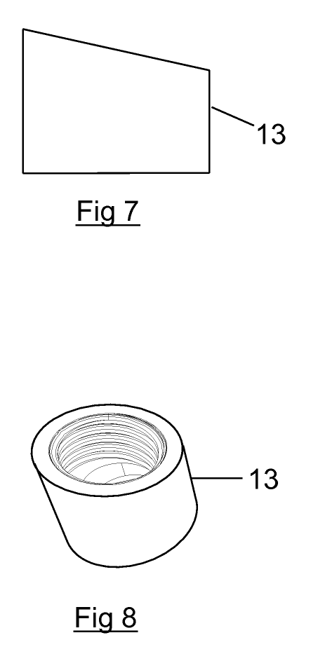

At the bottom of the bottle a threaded mounting 12 is formed, arranged to allow the bottle to be mounted on a corresponding internally threaded mounting base 13 as shown in FIGS. 7 and 8. Such mounting base arrangements are known, and therefore need not be described further.

The invention provides for convenient and economic manufacture of a liquid dispenser with air freshener. Use of a strip in conjunction with a shaped container to define a cavity to receive an air freshening substance obviates the need for an additional moulded component. The strip can easily be perforated as desired to present a chosen image.

The above embodiment is described by way of example only. Many variations are possible without departing from the invention as defined by the appended claims.

I claim:

1. A liquid dispenser comprising a container for receiving a liquid and a strip of sheet material secured to the container, the container being shaped such that the strip of sheet material and container together define an at least partially enclosed space in which an air freshening product may be disposed.

2. A liquid dispenser as claimed in claim 1, wherein the container is shaped to define a recess and the strip of sheet material overlies the recess.

3. A liquid dispenser as claimed in claim 1, wherein the strip of sheet material is perforated.

4. A liquid dispenser as claimed in claim 1, wherein the strip of sheet material is breathable.

5. A liquid dispenser as claimed in claim 3, wherein the perforations in the strip of sheet material form a word.

6. A liquid dispenser as claimed in claim 1, wherein an air freshening product is disposed in the at least partially enclosed space.

7. A liquid dispenser as claimed in claim 6, wherein the air freshening product comprises a strip of sheet material.

8. A liquid dispenser as claimed in claim 1, wherein the container is a bottle with a recess formed around its periphery and the strip of sheet material extends around the periphery of the bottle in the region of the recess.

9. A liquid dispenser as claimed in claim 1, wherein the strip of sheet material comprises a fastening means by which the strip may be secured to the container.

10. A liquid dispenser as claimed in claim 1, wherein the fastening means comprises cooperating engageable formations in the strip of sheet material enabling the strip to be fastened to itself thereby securing it to the container.

11. A liquid dispenser as claimed in claim 1 wherein a liquid soap product is disposed in the container.
